(19) United States
(12) Patent Application Publication
(10) Pub. No.: US 2008/0294106 A1
(43) Pub. Date: Nov. 27, 2008

(54) MULTIPLE ROBOTIC INJECTIONS OF ANESTHESIA BASED ON SCANNED IMAGE

(75) Inventor: Raymond H. Chen, San Marino, CA (US)

Correspondence Address: MCDERMOTT WILL & EMERY LLP, 2049 CENTURY PARK EAST, 38th Floor, LOS ANGELES, CA, 90067-3208, US

(73) Assignee: CONVERGENT MEDICAL SOLUTIONS, INC., San Marino, CA (US)

(21) Appl. No.: 12/127,667

(22) Filed: May 27, 2008

Related U.S. Application Data
(63) Continuation-in-part of application No. 11/752,143, filed on May 22, 2007.

(51) Int. Cl. A61M 5/00; A61B 8/00; A61B 19/00

(52) U.S. Cl. 604/116; 600/439; 606/130

(57) ABSTRACT

A system for relieving pain caused by a tumor within a body may include an injection system having an injector configured to inject an anesthetic within the body to relieve the pain caused by the tumor, and a robotic system configured to position the injector at a plurality of specified, spaced-apart injection locations within the body. A method of relieving pain caused by a tumor within a body may include: generating an image of the tumor within the body; identifying, based on the image, a plurality of locations within the body at which an injection of an anesthetic that relieves the pain caused by the tumor should be made; and making injections of the anesthetic at the locations, under the control of a robotic system that is programmed to make the injections at the identified locations without human intervention between the injections.

### CROSS-REFERENCE TO RELATED APPLICATION

This application is a continuation-in-part of U.S. patent application Ser. No. 11/752,143, filed on May 22, 2007, and entitled “Microscopic Tumor Injection Treatment,” the entire content of which is incorporated herein by reference.

### BACKGROUND

1. Field

This disclosure relates to medical treatments and to robotics.

2. Description of Related Art

Cancer is the second-leading cause of death in the United States. Death from cancer frequently involves the metastatic spread of the invading tumor, whose growth and invasion of the major organs eventually lead to organ failure and death.

Cancer is often painful, as a tumor's infiltration of the sensory nervous system in human organs usually leads to relentless and persistent pain. To lessen the pain, cancer patients traditionally receive escalating doses of pain medicine, such as morphine.

Unfortunately, ever-increasing doses of medicine fail to provide long-term relief, because a patient's body develops tolerance to the pain medicine. Also, the side effects of the medicine, such as depression, respiratory suppression and drowsiness, eventually preclude further escalation of the pain medicine for pain relief. Furthermore, since the source of pain is persistent and growing, pain does not decrease over time, as in the case of healing from a broken bone or a surgical incision, but rather continues to escalate as the cancer persistently infiltrates more and more parts of the body.

### SUMMARY

A system for relieving pain caused by a tumor within a body may include an injection system having an injector configured to inject an anesthetic within the body to relieve the pain caused by the tumor, and a robotic system configured to position the injector at a plurality of specified, spaced-apart injection locations within the body.

A method of relieving pain caused by a tumor within a body may include injecting, at one or more spaced-apart locations within the body, an anesthetic that relieves the pain caused by the tumor, wherein at least some of the locations may be within or in the vicinity of the tumor.

These, as well as other components, steps, features, objects, benefits, and advantages, will now become clear from a review of the following detailed description of illustrative embodiments, the accompanying drawings, and the claims.

### BRIEF DESCRIPTION OF DRAWINGS

The drawings disclose illustrative embodiments. They do not set forth all embodiments. Other embodiments may be used in addition or instead. Details that may be apparent or unnecessary may be omitted to save space or for more effective illustration. When the same numeral appears in different drawings, it is intended to refer to the same or like components or steps.

FIG. 1 illustrates an imaging system being used to generate an image of a targeted region within a body.

FIG. 2 illustrates a treatment plan being generated based on images that were generated by the imaging system illustrated in FIG. 1.

FIG. 3 illustrates a system for injecting injections within a body at multiple locations in accordance with a treatment plan.

### DETAILED DESCRIPTION OF ILLUSTRATIVE EMBODIMENTS

In the present disclosure, methods and systems are disclosed for medical treatments of a targeted region within the body based on robotic injections. In one embodiment, the targeted region may be a tumor, and the robotic injections may be performed for purposes of treating the tumor. In another embodiment, the targeted region may be a tumor, and the robotic injections may be performed for purposes of locally administering an anesthetic to relieve the pain caused by the tumor. In another embodiment, the targeted region may be a transplanted organ, and the robotic injections may be performed for purposes of delivering immunosuppressive drugs into the transplanted organ so as to suppress white cells infiltrating the transplanted organ without suppressing white cell activity throughout the rest of the body. In yet another embodiment, the targeted region may be an unborn fetus, and the robotic injections may be performed for purposes of delivering prenatal medications, thereby avoiding the detrimental consequences of postponing necessary treatments until birth. Many other embodiments are possible.

Illustrative embodiments are now discussed. Other embodiments may be used in addition or instead. Details that may be apparent or unnecessary may be omitted to save space or for a more effective presentation.

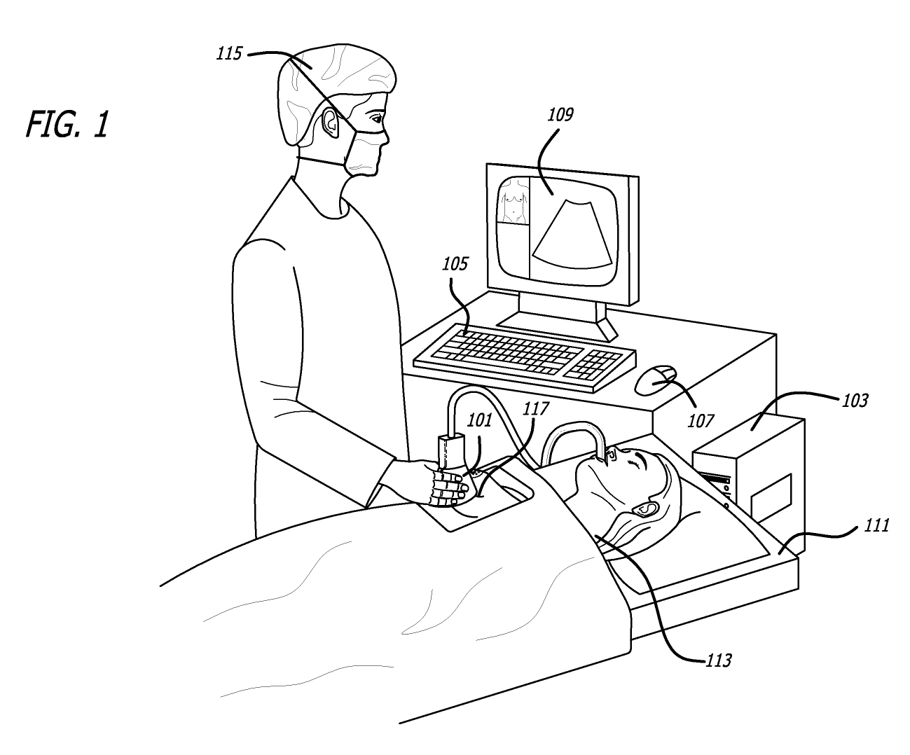

FIG. 1 illustrates an imaging system that is used to generate an image of a specific targeted region within a body, so that the targeted region may receive treatment. The targeted region may be, but is not limited to, a tumor, a transplanted organ (such as a transplanted liver, kidney, or heart, by way of example), and an unborn fetus. In the illustrated exemplary embodiment, the imaging system is an ultrasound imaging system that uses an ultrasound wand, and may be of any type, such as a GE Logiq 9 BTO4 3D/4D Ultrasound 2004 U/S Shared System.

As shown in FIG. 1, the imaging system may include an ultrasound wand 101, a processing system 103, a user interface that may include a keyboard 105, a mouse 107, a display 109, and/or any other type of device.

The hand-held ultrasound wand 101 may be of any type, such as a Phillips Sonos 7500 Echocardiography Ultrasound System/x4 Matrix Broadband phased array transducer.

The processing system 103 may include one or more microprocessors, storage devices, and input/output interface devices. It may be in one location or distributed across many locations. The processing system 103 may be part of a general purpose computer such as a PC or Mac or may be a special purpose computer dedicated to performing the functions described herein as well as others. It may be a stand alone system or part of a networked system. It may include software configured to cause the processing system 103 to perform one or more of the functions that are described herein, as well as other functions. The user interface may be used to controllably cause the processing system to perform one or more of the functions that are described herein.

A patient table 111 may be used to support a patient 113 so that the patient 113 is comfortable. Apparatus (not shown) may be provided in connection with the patient table to immobilize the patient or the portion of the patient that is imaged during the imaging process.

An equipment operator 115, such as a physician or a technician, may stand or sit next to the patient, hold the ultrasound wand 101, and operate them both so as to cause them to perform the functions described herein.

The treatment process may begin by having the patient 113 lie comfortably on the patient table 111. Adjustments may be made to restraining apparatus (not shown) to immobilize the patient or the portion of the patient that is being imaged, such as a breast.

The equipment operator 115 may place a mark 117 on the surface of skin on the patient 113 above the targeted region to be treated. Additional marks may be made elsewhere. The mark 117 may be made in any manner and/or with any material. The mark may be made in a way and/or with a material that ensures that the mark will remain on the patient's skin throughout the treatment process, even if this takes place over several days. The mark 117 may be made in a way and/or with a material that ensures that the mark 117 will appear in one or more images that will later be made.

In lieu of or in addition to the mark 117, a stabilizing device or platform may be used to secure the location of the imaging device.

The ultrasound wand 101 may be a hand-held unit as illustrated in FIG. 1 or may be mechanically linked to an arm whose position is sensed. If hand-held, the wand may be associated with a position-sensing systems that sense the position of the ultrasound wand 101 with respect to the patient table 111, the mark 117, and/or any other appropriate location.

The ultrasound wand 101 may be swept back and forth across the mark on the skin in multiple directions by the equipment operator 115, with appropriate tilting, so as to sweep out a volume containing the targeted region (and surrounding tissue when appropriate), such as a pyramid. This may performed in accordance with well-known ultrasound imaging techniques.

The processing system 103 may receive signals from the ultrasound wand 101 that are indicative of ultrasound reflections from tissue within the volume that is swept out by the ultrasound wand 101. The processing system 103 may generate an image of these reflections during each sweep and cause this image to display on the display 109 during each sweep. The equipment operator 115 may view these images during each sweep so as to insure that the sweep is being performed in the right area and at the right angle. Again, this may be done in accordance with well-known ultrasound imaging techniques.

The processing system 103 may store the signals that it receives from the ultrasound wand 101 and/or data that is derived from these signals. The processing system may generate and store three dimensional image data representative of the tissue within the volume that has been swept, including the targeted region. Again, this may be done in accordance with well-known ultrasound imaging techniques.

The ultrasound scanning technique that has just been described may be used in connection with targeted regions at any desired locations within the body. In the case of tumor treatment, for example, it may be used for tumors that are located close to the surface of the patient's body, such as a tumor in a breast. If the targeted region is a tumor that is located deeper in the body, such as a tumor in a liver, a laparoscopic ultrasound probe may be used instead, such as a B-K Medical Type 8666 Laparoscopic Ultrasound Transducer.

When a laparoscopic ultrasound probe is used, an incision may be made in the skin and abdominal wall above the targeted region. A probe port may be inserted through the incision in the skin and abdominal wall and directed toward the targeted region. An ultrasound probe with an ultrasound sensor at its end may be inserted into the probe port.

A camera port may similarly be inserted through the same or through a different incision that may be made through the skin and abdominal wall. A camera may then be inserted into the camera port.

A marker port may be inserted through one of these incisions or through a different incision that may be made through the skin or abdominal wall. A marker may be inserted into the marker port. The marker may be configured to make a mark that will last throughout the treatment process and will later be viewable with the camera. The marker may be a pen, cutter or burner. The marker may also be a stabilizing device or platform which can be used to secure the location of the ultrasound wand.

In an embodiment of the present disclosure in which the targeted region is a tumor, the cavity in which the tumor resides may be inflated with a gas, such as carbon dioxide. The camera may be activated and used to help guide the marker over the tumor on the surface of the organ in which the tumor resides. A mark on the surface of the organ above the tumor may then be made. The camera may be used to help guide the ultrasound probe to be positioned on the mark. The ultrasound probe may be swept back and forth across the mark on the skin in multiple directions, with appropriate tilting, so as to sweep out a volume containing the tumor and surrounding tissue, such as a pyramid. The camera may be used to help guide the ultrasound probe during the sweeps. This may all be performed in accordance with well-known ultrasound imaging techniques. The signals from the ultrasound probe may be processed by the processing system 103 as described above in connection with the ultrasound wand 101.

Other imaging systems may be used in addition or instead of the illustrated ultrasound imaging system. These imaging systems may include, but are not limited to, an MRI system, a CT scanning system, and/or a PET scanning system.

A treatment plan for the targeted region may be created next. In an embodiment of the present disclosure in which an image of the tumor is generated for purposes of treating the tumor, the treatment plan may include an identification of multiple locations within the body at which an injection that treats the tumor should be made. In an embodiment of the present disclosure in which an image of the tumor is generated for purposes of relieving pain caused by the tumor, the treatment plan may include an identification of multiple locations within the body at which an injection of an anesthetic that relieves and/or reduces the pain caused by the tumor should be made.

In an embodiment of the present disclosure in which an image of a transplanted organ is generated in order to deliver immunosuppressive drugs to the transplanted organ, the treatment plan may include an identification of one or more locations at which an injection of immunosuppressive drugs should be made in order to suppress infiltration of white cells into the transplanted organ as the body attempts to reject the transplanted organ.

In an embodiment of the present disclosure in which an image of an unborn fetus is generated in order to deliver prenatal medication(s) to the fetus, the treatment plan may include an identification of one or more locations at which an injection of prenatal medications should be made in order to provide necessary prenatal treatment to the fetus without postponing until birth.

The treatment plan may include information about the angle at which each injection should be made, so as to avoid unnecessary damage to vital organs. The angle for each injection may be different or the same.

In an embodiment in which tumors are identified for treatment, the identified locations may be anywhere with respect to the tumor. For example, they may be within the tumor and/or outside of the tumor. In some situations, for example, it may be desirable not to pierce the tumor but to make injections only in areas that surround it, such as approximately two to four centimeters beyond the exterior of the tumor. The locations may also be selected so as to avoid vital organs.

For tumor treatment, the identified locations may be spaced-apart by only small amounts. For example, the identified locations that neighbor one another may be spaced about by between 0.5 and 10 millimeters. Spacing in the range of 1 to 2 millimeters may also be used. The spacing between each set of neighboring locations may be the same or may be different.

For local delivery of a pain-relieving anesthetic to tumors, the identified locations may be within the tumor, and/or in the vicinity of the tumor, i.e. in areas that surround the tumor.

In an embodiment in which a transplanted organ is identified for delivery into the organ of immunosuppressive drugs, the identified locations may be within the transplanted organ, or in the areas in the vicinity of, the transplanted organ.

In an embodiment in which a fetus is identified for injection of prenatal medications, the identified locations may be within the fetus, or in the areas in the vicinity of the fetus. For example, prenatal medications may be injected in order to treat congenital defects. Traditionally, the placental barrier caused problems by preventing the medication taken by the mother from ever reaching the fetus. Using ultrasound imaging techniques as described above, fetal large vessels may be identified in order to allow direct medicinal injection into these vessels.

The injection locations may be specified in any way, such as by specifying a three dimensional coordinate relative to a fixed point, such as the marking that may have been made on the surface of the patient's skin or on the surface of an internal organ.

The treatment plan may specify the type of each treatment injection, as well as the dose of each injection. In the case of tumor treatment, for example, a chemotherapy agent, electromagnetic energy (such as energy in the RF, microwave and/or infrared range), and/or a cryotherapy agent may be specified as the injection type. The type of each injection may be the same or may be different at each location.

In the case of tumor pain relief, one or more anesthetics may be specified as the injection type. These anesthetics may include, but are not limited to, local anesthetics that have traditionally been clinically used, such as amino esters and amino amides. Any other types of injectable anesthetics may be used. These anesthetics may be injected in conjunction with injections of one or more chemotherapy agents to locally shrink the tumor. The pain relieving anesthetic may be selectively injected around each tumor at the leading edges of the cancer, where cancer cells infiltrate and compress normal tissues and their associated nerves.

In the case of immunosuppressive drug delivery, one or more immunosuppressive drugs may be specified as the injection type. These immunosuppressive drugs may include, but are not limited to: steroids such as cortisone; antilymphocyte serum; and analogs of purines and pyrimidines. These drugs may be injected into the transplanted organ once a rejection episode is diagnosed for the transplanted organ.

In the case of prenatal medication delivery, one or more prenatal medications may be injected into the fetus (for example into the fetal vessels), and/or into local tissue surrounding the fetus. As one example, described above, prenatal medications for curing congenital defects may be injected directly into the fetal vessels, thus overcoming the problems caused by the placental barrier which had prevented medication taken by the mother from reaching the fetus.

As another example, an HIV anti-viral medication may be injected into the fetus. When a healthy fetus is carried by a HIV+ mother, the fetus is often not infected by the HIV virus even when the mother carries the virus, due to the placental-barrier. The infection to the child occurs later when the child is born, as childbirth is the only time when the blood of mother and child mix. Prenatal injection of the HIV anti-viral medication prior to delivery can protect the fetus from the impending exposure to the HIV virus, and may be able to prevent HIV infection.

As another of many possible examples, a stem cell may be injected into a fetus with Severe Combined Immune Deficiency (SCID), before birth. A fetus with SCID has no immune system (such a fetus is commonly referred to as a “Boy in the bubble”). The injection of the stem cell into the body of am SCID fetus before birth may allow for proper maturation of the immune system, so that the baby will not be too vulnerable to the environmental contagions after birth.

The specifics of the dose specification may depend upon the type of injection. In the case of tumor treatment, for example, the dose may be specified as a volume of fluid, when a chemotherapy or cryotherapy agent is injected. When the tumor treatment injection is of electromagnetic energy, the dose may be specified by both a level of energy and the duration of its application. The dose may in addition or instead be specified in terms of a volume of tissue to be treated.

For tumor treatment, no matter how the dose is specified, the specification may be directly or indirectly selected so that each tumor treatment injection treats a microscopic volume of tissue that is substantially less than the volume of the tumor. This allows the treatment plan to be highly customized for each patient and highly localized to only the areas in need of treatment. The volume of tissue that should be treated, for example, may be less than one cubic millimeter.

In the case of tumor pain relief, the dose that is specified for the anesthetic injection may be substantially less than a therapeutically effective amount of an orally or intravenously administered pain relief medication. The reason is that by injecting locally to the pain site, a much lower dose is required to achieve the desired analgesic effect, compared to pain relief drugs that are orally or intravenously administered.

In the case of immunosuppressive drug delivery or prenatal medication delivery, the treatment plan may specify the requisite dose that is necessary to achieve the desired therapeutic effect.

Any means may be used to generate the treatment plan. The treatment plan may or may not be based on imaging information relating to the targeted region, such as the three-dimensional image data that was generated and stored during use of the imaging system discussed above in connection with FIG. 1.

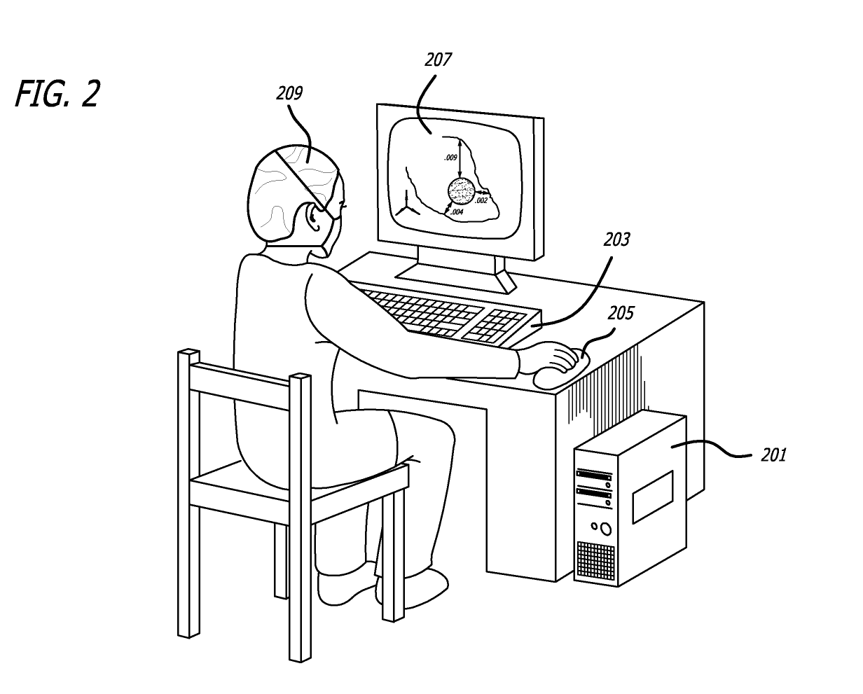

FIG. 2 illustrates a treatment plan being generated based on images that were generated by the imaging system illustrated in FIG. 1. As illustrated in FIG. 2, the treatment plan may be generated through the use of a processing system 201 and a user interface that may include a keyboard 203, a mouse 205, a display 207, and/or any other type of device.

The processing system 201 and user interface may be the same processing system and user interface that is illustrated in FIG. 1 or they may in whole or in part be different. If different, the processing system 201 may be any of the types and may contain any of the components or configurations that were discussed above in connection with the processing system 103 in FIG. 1. It may contain software configured to cause the processing system 201 to perform the functions described herein as well as others. The user interface may be used to controllably cause the processing system to perform one or more of the functions that are described herein.

An equipment operator 209, such as the same physician or technician that used the imaging system discussed above in connection with FIG. 1, or a different one, may stand or sit next to the user interface and operate it so as to cause the processing system 103 to generate the treatment plan.

The equipment operator 209 may study three-dimensional image data relating to the targeted region, such as the three-dimensional image data that that was generated by the imaging system shown in FIG. 1 and discussed above. The equipment operator 209 may demarcate the targeted region in the image using the keyboard 203, the mouse 205, the display 207, and/or any other means. The equipment operator may do so, for example, by clicking on various points on the periphery of the targeted region in the displayed image, thus establishing dimensional and location data for the targeted region. Pattern recognition technology may in addition or instead be used to demarcate the targeted region in whole or in part.

The equipment operator 209 may then specify the treatment plan based on the demarcated targeted region, such as the location of the various injections relative to a marker, the angle of each injection, the type of each injection, and/or the dose of each injection.

The processing system 201 may be configured to assist the equipment operator 209 with this task by generating the treatment plan in whole or in part from the demarcated image. For example, the equipment operator may merely specify the type of the injection, the volume to be treated by each injection, whether injections are to be made within or outside the targeted region, and the distance beyond the targeted region that is to be treated. The equipment operator may also demarcate areas within the body that are not to receive an injection or that must be avoided be the injection process. The processing system 201 may then calculate the treatment plan from this data, including the dose and location of each injection.

The processing system 201 may have an expert database and an associated expert system that enables the processing system 201 to generate the treatment plan based on the limited information that the equipment operator 209 has provided and the imaging data.

In the case of tumor treatment, the treatment plan may ultimately specify dozens or even hundreds of closely-spaced injections, each configured to treat only a very small volume of tissue.

In the case of tumor pain relief, the treatment plan may specify selected locations within and/or around the tumor, for example at the leading edges of the cancer, where cancer cells infiltrate and compress normal tissues and their associated nerves.

The processing system 201 may be configured to store this treatment plan for later use. It may associate the treatment plan with the identity of the patient 113, thus allowing the processing system 201 to be used for formulating the treatment plan of many patients.

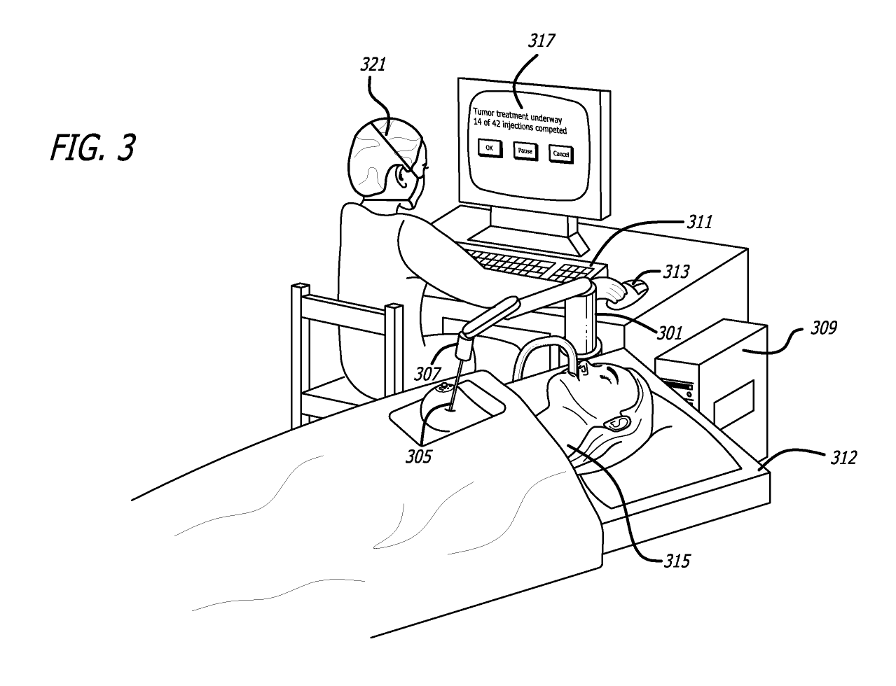

FIG. 3 illustrates an injection system for injecting injections within a body at multiple locations in accordance with a treatment plan. Any other means may be used in addition or instead to implement the specified treatment plan.

As shown in FIG. 3, the treatment system may include an injection system 301 that includes an injector 305 attached to a robotic system 307. The robotic system may be controlled by a processing system 309 that may be associated with a user interface, such as a keyboard 311, a mouse 313, a display 317, and/or any other type of device.

Depending on the targeted region, and the treatment being performed, the injector 305 may be configured to inject an injection that achieves a desired therapeutic effect, including but not limited to treating cancer, relieving pain, suppressing immune reactions, and delivering prenatal medication(s). For tumor treatment, the injection may consist of or include a chemotherapy agent, electromagnetic energy (such as energy in the R.F., microwave and/or infrared range), and/or a chyotherapy agent. For tumor pain relief, the injection may consist of or include an anesthetic. A chemotherapy agent may also be injected in conjunction with the anesthetic. For immunosuppressive treatment of transplanted organs, the injection may consist of or include immunosuppressive drugs. For prenatal treatment of unborn fetuses, the injection may be prenatal medications that include, but are not limited to: medications for treating congenital fetal defects; HIV anti-viral medications; and stem cells.

The injector 305 may be configured so as to allow the dose of the injection to be controlled. In the case of tumor treatment, the dose of the injection may include the volume of chemotherapy or chyotherapy agent that is injected, or the strength and duration of any electromagnetic energy that is injected. The injector 305 may be configured so as to provide a tumor treatment dose that treats only a microscopic volume of tissue, such as a volume that is less than one cubic millimeter.

In the case of tumor pain relief, the dose of the injection may include the volume of the anesthetic, and of the chemotherapy agent (if delivered in conjunction with the anesthetic). The injector 305 may be configured so as to provide an anesthetic treatment dose that is substantially less than a therapeutically effective amount of an orally or an intravenously administered pain relief medication.

In the case of transplanted organs or fetuses, the injector 305 may be configured to as to provide a treatment dose of the medication being injected that is necessary to achieve the desired therapeutic effect.

The injector 305 may include any type of injection device, such as a hypodermic needle, an air-powered injector, one or more electrodes, any type of electromagnetic radiation device, and/or any other type of injection device.

The robotic system 307 may be configured so that it can automatically position the injector at any specified location with the body of a patient 315 that may be resting on a patient table 312. The robotic system 307 may be configured so that it can position the injector above any surface area of the patient 315 and so that it can push the injector 305 downwardly into the patient to any specified depth. The robotic system 307 may be configured so that it can cause the injection to be made at any specified angle with respect to the surface of the patient 315.

The robotic system may be configured so that it can accurately make injections at specified, closely-spaced locations in three-dimensional space, such as at locations that are spaced apart by only between 0.5 and 10 millimeters.

The processing system 309 and user interface may be the same processing system and user interface that is illustrated in FIG. 1 or 2 or they may in whole or in part be different. If different, the processing system 201 may be any of the types and may contain any of the components or configurations that were discussed above in connection with the processing system 103 in FIG. 1. The processing system 103 may contain software configured to cause it to perform the functions described herein as well as others. The user interface may be used to controllably cause the processing system to perform one or more of the functions that are described herein.

The processing system 309 may be configured to control the positioning of the robotic system 307. For example, the processing system may be configured to cause the robotic system 307 and, in turn, the injector 305, to sequentially move to the various locations specified by the treatment plan without intervention from a human between each move.

Similarly, the processing system 309 may be configured to cause the injector 305 to inject an injection that delivers a treatment at each specified location, again in accordance with the treatment plan and without intervention from a human between each injection.

A patient table 319 may be provided to keep the patient 315 comfortable during the treatment. The patient table may include restraining apparatus (not shown) to immobilize the patient or the treated portion of the patient during treatment. The patient table 319 may be the same as the patient table 111 that is shown in FIG. 1 or may be different.

An equipment operator 321 may be present to operate the treatment system. The equipment operator 321 may be a physician or a technician or any other person. It may be the same person as the equipment operator 115 that is illustrated in FIG. 1, the equipment operator 209 that is illustrated in FIG. 2, or may be a different person.

The treatment system that is illustrated in FIG. 3 may be used to implement any process. For example, the patient 315 may be placed on the patient table 319 and the breast of the patient may be immobilized. The robotic system 307 may be controlled by the equipment operator 209 so as to cause the injector 305 to touch the mark 117 that was previously made on the patient 315. The equipment operator may then signal the processing system 309 that the injector 305 is in contact with the mark 117, thus registering the coordinate system of the robotic system with the coordinate system of the imaging system that is shown in FIG. 1 and discussed above.

Any other means of registration may be used in addition or instead. For example, the robotic system 307 may be mechanically linked to the ultrasound wand 101 shown in FIG. 1 or the ultrasound probe that was discussed above, thus making registration automatic.

Following registration, the equipment operator 321 may initiate the treatment plan by appropriate commands to the processing system 309 through the user interface. Thereafter, the processing system 309 may implement the treatment plan by causing the robotic system 307 to position the injector 305 at each of the treatment locations that are specified in the treatment plan and to provide an injection at that location to treat the targeted region, also in accordance with the treatment plan. The processing system 309 may cause the robotic system 307 to move sequentially to each treatment location and cause the injector 305 to make each desired injection at each location, all without intervention from a human in between each injection.

For example, in the case of breast tumor treatment, the processing system 309 may direct the robotic system 307 to move the injector 305 immediately above the surface of the breast of the patient 315 where the first injection is to be made. The processing system 309 may instead direct the robotic system 307 to angle the injector 305 and to offset it from the position immediately above the first injection point to compensate for the angle. The processing system 309 may then direct the robotic system 307 to push the injector 305 through the surface of the skin of the patient 315 to the first injection location and may then cause the injector 305 to make a specified injection at the treatment location.

The processing system 309 may then cause the robotic system 307 to move the injector 305 to a different depth within the patient 315, but without removing the injector 305 from the patient. The processing system may then direct the injector 305 to make a second injection, again in accordance with the treatment plan. Thereafter, the processing system 309 may direct the robotic system 307 to withdraw the injector 305 from the patient, to position the injector 305 above a different site, and to implement a further injection regiment at that different location. This process may repeat until the entire treatment plan has been performed, all without human intervention between each injection.

The injector 305 may instead operate through an injector port, much in the same way as a laparoscopic ultrasound probe operates in conjunction with a probe port, as discussed above in connection with FIG. 1. The injector may operate through the same probe port that was inserted during the imaging or may operate through a different port. Similarly, the same or different camera may be used through the same or different camera port to aid in registering the injector 305 to a mark on the surface of an internal organ. Air, such as carbon dioxide, may first be injected to inflate the abdominal area so that the camera can be free to aid in this registration. After registration is effectuated, the equipment operator 321 may similarly direct the processing system 309 to implement the treatment plan, again without human intervention between injections. During this implementation, however, the injector 305 may not be fully withdrawn from the patient until after the sequence of injections is complete. Instead, the injector 305 may remain within the port throughout the treatment. Still, it may be withdrawn from the organ and then repositioned above a different surface location on the organ between certain injections.

An imaging system may be added and configured to display images of the injector 305 with respect to the tumor before or during implementation of the treatment plan. This may aid the equipment operator 321 in verifying that the injections are being made at the appropriate locations.

Although having thus-far been described as making a single injection at any one point in time, the injector 305 may include a plurality of injection devices, such as a plurality of hypodermic needles, air-powered injectors, electrodes, and/or electromagnetic radiation devices that may controllably make multiple injections simultaneously. These multiple injection devices may be arranged in straight line, in a two dimensional array, or even in a three-dimensional array. In the case of tumor treatment, Neighboring injectors may be closely spaced, such as between 0.5 and 10 millimeters apart. In this embodiment, the processing system 309 may be configured to direct the injection system 301 to make multiple injections simultaneously, thus reducing the number of movements the robotic system 307 may need to make to fully implement a treatment plan.

The robotic injections of anesthesia, described above, may provide pain relief to the cancer patients at much lower doses, compared to orally delivered pain relief medication such as morphine. By starting with a lower effective dose, more room may be allowed for dosage escalation later, thus decreasing the chance that a terminal patient will reach the maximal allowable dosage before the end of life. Moreover, targeted injection at the tumor itself may avoid systemic side effects of pain medicine, including but not limited to depression, respiratory suppression and drowsiness, which occur when the medicine reaches the brain.

The robotic injections of immunosuppressive drugs, described above, may allow immunosuppressive regimens to be injected directly into the transplanted organ. In this way, the injected immunosuppressive medicine may suppress the white cells infiltrating the transplanted organ and stop the attack on said transplanted organ. Because no systemic immunosuppressive medications are delivered, either orally or intravenously, white cells throughout the rest of the body can continue performing their duties in preventing infection.

The robotic injections of prenatal medication into unborn fetuses, as described above, allow for precise, minimally-invasive pre-natal intervention in procedures for which pre-natal intervention is currently not available. These procedures include, but not limited to: treatment of congenital defects; delivery of HIV anti-viral medication; and injection of stem cells for fetuses that lack an immune system.

The components, steps, features, objects, benefits and advantages that have been discussed are merely illustrative. None of them, nor the discussions relating to them, are intended to limit the scope of protection in any way. Numerous other embodiments are also contemplated, including embodiments that have fewer, additional, and/or different components, steps, features, objects, benefits and advantages. The components and steps may also be arranged and ordered differently.

The phrase “means for” when used in a claim embraces the corresponding structures and materials that have been described and their equivalents. Similarly, the phrase “step for” when used in a claim embraces the corresponding acts that have been described and their equivalents. The absence of these phrases means that the claim is not limited to any of the corresponding structures, materials, or acts or to their equivalents.

Nothing that has been stated or illustrated is intended to cause a dedication of any component, step, feature, object, benefit, advantage, or equivalent to the public, regardless of whether it is recited in the claims.

In short, the scope of protection is limited solely by the claims that now follow. That scope is intended to be as broad as is reasonably consistent with the language that is used in the claims and to encompass all structural and functional equivalents.

While the specification describes particular embodiments of the present invention, those of ordinary skill can devise variations of the present invention without departing from the inventive concept.

I claim:

1. A system for relieving pain caused by a tumor within a body, comprising:
an injection system having an injector configured to inject an anesthetic within the body that relieves the pain caused by the tumor; and
a robotic system configured to position the injector at one or more specified, spaced-apart injection locations within the body.

2. The system of claim 1 further comprising a processing system configured to provide information to the robotic system indicative of the injection locations.

3. The system of claim 2 wherein the processing system is configured to cause the robotic system to sequentially position the injector at a plurality of the injection locations and to cause the injection system to inject the anesthetic at each of the injection locations, all without human intervention between the injections of the anesthetic.

4. The system of claim 1 wherein at least one of the injection locations is within the tumor.

5. The system of claim 1 wherein at least one of the injection locations is in the vicinity of the tumor.

6. The system of claim 3 wherein the processing system is configured to provide information to the injection system indicative of the dose of the anesthetic injected at each injection location.

7. The system of claim 3 further comprising an imaging system configured to provide an image of the tumor in the body, wherein the processing system is configured to provide the information indicative of the injection locations based on the image provided by the imaging system.

8. The system of claim 7 wherein the imaging system is a three-dimensional ultrasound imaging system.

9. The system of claim 7 wherein the imaging system is an MRI imaging system.

10. The system of claim 7 wherein the imaging system is a CT (computerized tomography) imaging system.

11. The system of claim 7 wherein the imaging system is a PET scanning system.

12. The system of claim 7 wherein the imaging system is configured to provide an image of the injector relative to the tumor while the injector is in the body.

13. The system of claim 1 wherein the injector includes a plurality of hypodermic needles.

14. The system of claim 1 wherein the injector is further configured to inject a chemotherapy agent at one or more treatment locations within the body to reduce the size of the tumor.

15. The system of claim 1 wherein the injection system is configured to inject an injection within the body that is dosed to deliver an amount of anesthetic that is substantially less than a therapeutically effective amount of an orally or an intravenously administered pain relief medication.

16. A method of relieving pain caused by a tumor within a body comprising injecting, at one or more spaced-apart locations within the body, an anesthetic that relieves the pain caused by the tumor, wherein at least some of the locations are within or in the vicinity of the tumor.

17. The method of claim 16, wherein the injections are sequentially made without human intervention between each injection.

18. A method of relieving pain caused by a tumor within a body comprising:
generating an image of the tumor within the body;
identifying, based on the image, a plurality of locations within the body at which an injection of an anesthetic that relieves the pain caused by the tumor should be made; and
making injections of the anesthetic at the locations, under the control of a robotic system that is programmed to make the injections at the identified locations without human intervention between the injections.

19. The method of claim 18, wherein the act of generating an image of the tumor within the body comprises at least one of:
generating an ultrasound image of the tumor;
generating a CT (computerized tomography) image of the tumor;
generating an MRI of the tumor; and
generating a PET scan of the tumor.
